(19) United States
(12) Patent Application Publication
(10) Pub. No.: US 2002/0127273 A1
(43) Pub. Date: Sep. 12, 2002

(54) Liposomal nystatin treatment of fungal infection

(75) Inventor: Thomas Walsh, Bethesda, MD (US)

Correspondence Address: Thomas M. Saunders, Lorusso & Loud, 440 Commercial Street, Boston, MA, 02109, US

(73) Assignee: Aronex Pharmaceuticals, Inc.

(21) Appl. No.: 09/950,980

(22) Filed: Sep. 11, 2001

Related U.S. Application Data
(60) Provisional application No. 60/232,744, filed on Sep. 15, 2000.

(51) Int. Cl. A61K 31/7048; A61K 9/127

(52) U.S. Cl. 424/450; 514/28

(57) ABSTRACT

A method of treating a urinary tract fungal infection in a human comprising systemically administering a therapeutically effective amount of a liposomal polyene including nystatin and amphotericin wherein the fungal infection is selected from the group consisting of aspergillosis, candidiasis (e.g., C. parapsilosis, C. albicans, C. tropicalis, C. glabrata, C. lusitaniae), zygomycosis, cryptococcosis, histoplasmosis, blastomycosis, cladosporiosis, fusariosis, Bipolaris hawaiiensis, Dactylaria gallopava, torulopsosis, Acremonium kiliense, Cryptococcus neoformans, and Histoplasma capsulatum.

### STATEMENT OF GOVERNMENT RIGHTS

The U.S. Government has a paid-up license in this invention and the right in limited circumstances to require the patent owner to license others on reasonable terms as provided for by the terms of National Cancer Institute Grant No. ______.

### FIELD OF THE INVENTION

A method of treating a urinary tract fungal infection in a human comprising systemically administering a therapeutically effective amount of a liposomal polyene including nystatin and amphotericin wherein the fungal infection is selected from the group consisting of aspergillosis, candidiasis (e.g., C. parapsilosis, C. albicans, C. tropicalis, C. glabrata, C. lusitaniae), zygomycosis, cryptococcosis, histoplasmosis, blastomycosis, cladosporiosis, fusariosis, Bipolaris hawaiiensis, Dactylaria gallopava, torulopsosis, Acremonium kiliense, Cryptococcus neoformans, and Histoplasma capsulatum.

### BACKGROUND OF THE INVENTION

The polyene macrolide antibiotics are secondary metabolites produced by various species of Streptomyces. Several common features of these compounds are useful in classifying the more than 80 different polyenes that have been isolated. All are characterized by a macrolide ring, composed of 26-38 carbon atoms and containing a series of unsaturated carbon atoms and hydroxyl groups. These features of the molecule contribute to the polyenes' amphipathic properties (those relating to molecules containing groups with different properties, for example, hydrophilic and hydrophobic). The ring structure is closed by the formation of an internal ester or lactone bond. The number of conjugated double bonds vary with each polyene, and the compounds are generally classified according to the degree of unsaturation.

Toxic effects of polyene macrolides appear to be dependent on binding to cell membrane sterols. Thus, they bind to membranes of fungus cells as well as to those of other eukaryotic cells (human, plant, and protozoa), but not to bacterial cell membranes, which do not contain membrane sterols. The interaction of polyene macrolides with mammalian and fungal membrane sterols results in transmembrane channels that allow the leakage of intracellular components leading to cell deaths.

Nystatin (C47H75NO17; mol.wt. 926.13), discovered as the first antifungal polyene antibiotic in the early 1950s, is a macrocyclic lactone consisting of a hydroxylated tetraene diene backbone and a mycosamine residue. Similar to amphotericin B, nystatin has potent and broadspectrum fungicidal activity in vitro. However, early problems with solubilization and toxicity after parenteral administration precluded the compound's use for systemic treatment.

A multilamellar liposomal formulation consisting of dimyristoyl phosphatidylcholine (DMPC), dimyristoyl phosphatidylglycerol (DMPG) and nystatin in a 7:3:1 molar ratio and a particle-size of 0.1-3μm is noted. This liposomal formulation has been shown to have reduced toxicity to mammalian cells but preserved in vitro antifungal activity and it has demonstrated encouraging activity in animal models of opportunistic fungal infections. It is well tolerated in patients without dose-limiting toxicity at dosages of up to 8 mg/kg/day and has shown efficacy in non-neutropenic patients with candidemia. Dosages of 2 and 4 mg/kg/day are particularly noted.

### SUMMARY OF THE INVENTION

A study of plasma pharmacokinetics and tissue distribution of multilamellar liposomal nystatin in normal rabbits, established surprisingly high concentrations of nystatin in spot urine. This finding was further examined as to disposition of liposomal polyene by means of urinary pharmacokinetics and drug disposition in comparison to amphotericin B deoxycholate as the standard agent. High polyene concentrations ion the urinary tract are particularly useful in immunocompromised subjects or subjects with an obstructed urethra wherein the subjects require proximal diversion of urine and indwelling stents.

This invention comprises a method of treating a urinary tract fungal infection in a human comprising systemically administering a therapeutically effective amount of a liposomal polyene, with particular reference to the polyenes amphotericin B and nystatin. In certain embodiments the method will include administering the therapeutically effective amount for at least about 5 days. Particular reference is made to an administered therapeutically effective amount of liposomal polyene of from about 1 mg/kg to about 8 mg/kg/day. In specific embodiments the therapeutically effective amount of administered liposomal polyene is a sub-MED amount. Reference is made to the polyene, nystatin, and the sub-MED amount of from about 1 mg/kg to 1.5 mg/kg/day, further including administering the sub-MED amount for at least about 5 days.

The method further contemplates a therapeutically effective amount as measured in peak plasma concentration of at least about 10 μg/mL, and at least about 13 μg/mL.

Noted is the method wherein the therapeutically effective amount is measured in AUC at μg/mL/hr is at least about 29 μg/mL/hr, and further at least about 52 μg/mL/hr.

In treating urinary tract infections by the claimed method, in specific embodiment, a therapeutically effective amount is as measured in glomerular filtrate.

The invention further comprises a method of treating a urinary tract fungal infection in a human comprising systemically administering a therapeutically effective amount of a liposomal polyene including nystatin and amphotericin wherein the fungal infection is selected from the group consisting of aspergillosis, candidiasis (e.g., C. parapsilosis, C. albicans, C. tropicalis, C. glabrata, C. lusitaniae), zygomycosis, cryptococcosis, histoplasmosis, blastomycosis, cladosporiosis, fusariosis, Bipolaris hawaiiensis, Dactylaria gallopava, torulopsosis, Acremonium kiliense, Cryptococcus neoformans, and Histoplasma capsulatum.

### BRIEF DESCRIPTION OF THE FIGURES

FIG. 1 (a) presents pharmacokinetic parameters of liposomal nystatin in plasma and urine arising from dosing at 2 mg/kg.

FIG. 1 (b) presents pharmacokinetic parameters of liposomal nystatin in plasma 5 and urine arising from dosing at 4 mg/kg.

FIG. 1 (c) presents pharmacokinetic parameters of liposomal nystatin in plasma and urine arising from dosing at 6 mg/kg.

FIG. 1 (d) presents pharmacokinetic parameters of Amphotericin B in plasma and urine arising from dosing at 1 mg/kg.

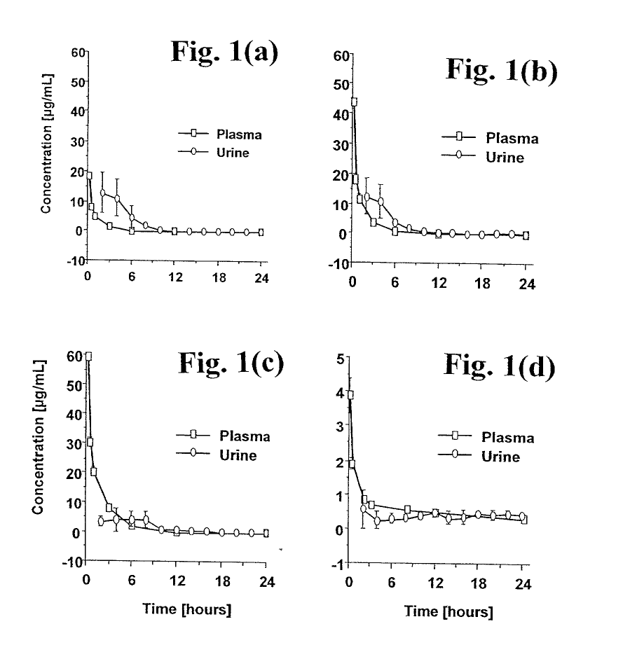

### DETAILED DESCRIPTION OF THE INVENTION

This invention will be better understood with reference to the following definitions:

A. Fungal agents shall be broadly understood to included any and all fungal agents with particular reference to causative agents in aspergillosis, candidiasis (e.g., C. parapsilosis, C. albicans, C. tropicalis, C. glabrata, C. lusitaniae), zygomycosis, cryptococcosis, histoplasmosis, blastomycosis, cladosporiosis, fusariosis, Bipolaris hawaiiensis, Dactylaria gallopava, torulopsosis, Acremonium kiliense, Cryptococcus neoformans, and Histoplasma capsulatum.

B. “Focus-therapeutic effect” in the context of this invention shall mean that a dosage of drug delivered systemically (i.e.,intravenously) results in systemic drug concentrations below a systemic minimum effective dosage (MED). However, in particular instances, such doses will, nevertheless, result in drug levels at, or in excess of, the minimum inhibitory concentration (MIC) in the glomerular filtrate within the kidney, or in the urine in the ureter, and bladder. A systemic dosage below the MED that results in a localized concentration in excess of the MIC in the kidney, ureter, and bladder is termed a “sub-MED” dose.

For a specific formulation of liposomal nystatin, a systemic dosage of about 1 mg to about 8 mg/kg/day provides a focused-therapeutic effect in the urinary tract. Particular reference is made to doses of from about 1 to 1.5 mg/kg/day, a sub-MED dose. Additional note is made of doses of from about 1.6 to about 2 mg/kg/day, and from about 2 to about 4 mg/kg/day. Dosage regimens lasting about 5 or more days are noted, with particular reference to regimens of from about 10 to about 30 days. In chronically immunocompromised patients, such as transplant patients taking imunosuppressive drugs, or in the case of HIV positive subjects, chronic dosing is anticipated, with particular reference to chronic sub-Med dosing.

Drug levels similar to liposomal nystatin are anticipate liposomal amphotericin B, with particular reference to doses of from about 1 to 1.5 mg/kg/day, a sub-MED dose.

It is recognized that in particular instances, renal polyene clearance by a damaged kidney will alter the concentration attained in the kidney, ureter and bladder at a given systemic dosage.

C. The kidney, ureter and bladder shall be termed “urinary tract.”

D. Liposomal polyene shall be broadly understood to encompass all lipid associated polyene dosage forms. Reference is made to liposomal nystatin as disclosed in U.S. Pat. No. 4,812,312 to Lopez-Berestein et al. and U.S. Pat. No. 5,178, 875 to Lenk et al., the teachings of which are incorporated herein by reference. Also included in the term “liposomal polyene” are other lipid polyene dosage forms such as those disclosed in U.S. Pat. No. 5,965,156, “Amphotericin B liposome preparation,” and U.S. Pat. No. 4,663,167 “Composition and method for treatment of disseminated fungal infections in mammals,” the teachings of which are incorporated herein by reference

For convenience, the term “liposomal polyene” shall extend to high drug:lipid complexes of polyenes such as amphotericin to those disclosed in U.S. Pat. No. 5,616,334 (Janoff et al) that, structurally, are not classical liposomes.

E. Therapeutically effective amount as to a drug dosage, shall mean that dosage that provides the specific pharmacological response for which the drug is administered in a significant number of subjects in need of such treatment. It is emphasized that drug resistance is a known problem in treating fungal infections. Thus, different strains of a single fungal genus or species present differing susceptibility to given drugs. Reference to “specific pharmacological response for which the drug is administered in a significant number of subjects in need of such treatment” is a recognition that a “therapeutically effective amount,” administered to a particular subject in a particular instance will not universally end a fungal infection in a particular subject, even though such dosage is deemed a “therapeutically effective amount” by those skilled in the art.

In comparison to the standard dosage of D-AmB of 1 mg/kg, L-Nys at the anticipated therapeutic dosage range of from about 2 to 6 mg/kg or more reveals a significantly faster clearance from plasma with complete elimination within the dosing interval and an at least sixfold smaller volume of distribution. Urinary data demonstrated at least tenfold higher Cmax and at least fourfold higher AUC24h values after administration of L-Nys. There was a trend toward a dose-dependent decrease in the renal clearance of L-Nys, suggesting dose-dependent functional effects or saturable tubular excretion. Independent of the dosage of L-Nys, at 24 hrs, less than 0.5% of the total dose was recovered from liver, spleen, kidneys and lung. In contrast, after D-AmB, 25% of the dose was found in the liver. The recovery of L-Nys in urine decreased with increasing dosages, but was not significantly different from that of D-AmB, suggesting that the differences in the disposition of both compounds may be explained by fundamental differences in biliary excretion or hepatic/extrahepatic metabolism.

Pharmacokinetic parameters of liposomal nystatin and amphotericin B in plasma are set forth in Table 1. Table 2 presents pharmacokinetic parameters of liposomal nystatin and amphotericin B in urine.

TABLE 1
Pharmacokinetic Parameters of L-Nys and D-AmB in Plasma:

| Drug/Dose | Cmax¹ | AUC0-24 h² | VD³ | CI⁴ | T½⁵ |
|---|---|---|---|---|---|
| [mg/kg] | [μg/mL] | [μg/mL · h] | [L/kg] | [L/h/kg] | [hrs] |
| L- | 17.00 ± 0.62 | 17.08 ± 0.48 | 0.155 ± 0.003 | 0.1173 ± 0.003 | 1.10 ± 0.00 |
| Nys | | | | | |
| 2 | | | | | |
| L-Nys | 39.27 ± 0.79 | 42.74 ± 5.25 | 0.138 ± 0.003 | 0.0928 ± 0.012 | 1.26 ± 0.09 |
| 4 | | | | | |
| L-Nys | 56.01 ± 0.42 | 77.12 ± 9.65 | 0.145 ± 0.002 | 0.0805 ± 0.010 | 1.44 ± 0.18 |
| 6 | | | | | |
| D- | 3.36 ± 0.42 | 12.23 ± 0.95 | 1.200 ± 0.043 | 0.0552 ± 0.007 | 16.82 ± 1.60 |
| AmB | | | | | |
| 1 | | | | | |

¹p < 0.001 for the comparison between L-Nys 2, 4, and 6 (Kruskal-Wallis ANOVA) and p < 0.001 for the comparison of L-Nys 2, 4, and 6 with D-AmB 1 (T-Test);
²p < 0.005 and p < 0.05;
³n.s. and p < 0.001;
⁴n.s. and p = 0.01 for Nys-2 vs. D-AmB 1;
⁵n.s. and p = 0.01.

TABLE 2
Pharmacokinetic Parameters of L-Nys and D-AmB in Urine

| Drug/ | | | | |
|---|---|---|---|---|
| Dose | Cmax U ¹ | Tmax U ² | AUCO-24 h U ³ | Clrenal ⁴ |
| [mg/kg] | [μg/mL] | [h] | [μg/mL · h] | [L/h/kg] |
| L-Nys 2 | 16.83 | 3.33 | 63.12 | 0.971 |
| | ± | ± | ± | ± |
| | 3.54 | 1.33 | 18.84 | 0.146 |
| L-Nys 4 | 18.52 | 2.67 | 60.71 | 0.434 |
| | ± | ± | ± | ± |
| | 4.85 | 0.66 | 8.68 | 0.307 |
| L-Nys 6 | 10.00 | 6.00 | 35.27 | 0.151 |
| | ± | ± | ± | ± |
| | 0.89 | 1.15 | 6.89 | 0.096 |
| D-AmB 1 | 0.962 | 12.67 | 8.95 | 0.004 |
| | ± | ± | ± | ± |
| | 0.352 | 6.36 | 3.14 | 0.001 |

¹ n.s. for the comparison between L-Nys 2, 4 and 6 (Kruskal Wallis ANOVA) and pd 0.05 for the comparison of L-Nys 2, 4 and 6 with D-AmB 1 (t Test).
² n.s. and n.s.;
³ n.s. and n.s., <0.05 and n.s.;
⁴ n.s. and <0.05, n.s., n.s.;

Table 3 presents tissue concentrations of liposomal nystatin and amphotericin B 24 hrs after dosing. Table 4 presents recovery of liposomal nystatin and amphotericin B from tissues and fluids 24 hrs after dosing

TABLE 3
Tissue Concentrations of L-Nys and D-AmB 24 hrs after Dosing

| | Concentration [μg/g or μg/mL] | | | |
|---|---|---|---|---|
| | L-Nys 2 | L-Nys 4 | L-Nys 6 | D-AmB 1 |
| Liver ¹ | 0.16 ± 0.00 | 0.26 ± 0.03 | 0.28 ± 0.10 | 7.04 ± 1.06 |
| Spleen ² | 0.39 ± 0.08 | 0.81 ± 0.12 | 0.86 ± 0.10 | 8.57 ± 0.06 |
| Kidney ³ | 0.39 ± 0.00 | 1.15 ± 0.14 | 1.59 ± 0.24 | 1.19 ± 0.10 |
| Lung ⁴ | 0.18 ± 0.00 | 0.33 ± 0.01 | 0.44 ± 0.03 | 1.10 ± 0.22 |
| Muscle | n.d. | n.d. | n.d. | 0.02 ± 0.00 |
| Fat | n.d. | n.d. | n.d. | n.d. |
| Brain | n.d. | n.d. | n.d. | 0.02 ± 0.00 |
| Bile | 1.63 ± 0.41 | 1.67 ± 0.26 | 2.10 ± 1.08 | 1.85 ± 0.43 |
| Urine | n.d. | 0.10 ± 0.10 | n.d. | 0.43 ± 0.06 |
| Plasma | n.d. | n.d. | n.d. | 0.25 ± 0.03 |

¹ <0.05 for the comparison between L-Nys 2, 4, and 6 (Kruskal-Wallis ANOVA) and p < 0.05 for the comparison of L-Nys 2, 4, and 6 with D-AmB 1 (T-Test);
² p = 0.05 and p = 0.005;
³ p < 0.005 and p = 0.01, 0.83, 0.27;
⁴ p < 0.005 and p = 0.05, 0.07, 0.10.
n.d., not detectable.

TABLE 4
Recovery of L-Nys and D-AmB from Tissues
and Fluids 24 hrs after Dosing

Recovery [% of Total Dose]

| | L-Nys 2 | L-Nys 4 | L-Nys 6 | D-AmB 1 |
|---|---|---|---|---|
| Liver ¹ | 0.285 | 0.248 | 0.180 | 25.87 |
| | ± | ± | ± | ± |
| | 0.005 | 0.031 | 0.021 | 3.570 |
| Spleen ² | 0.125 | 0.085 | 0.069 | 0.351 |
| | ± | ± | ± | ± |
| | 0.064 | 0.014 | 0.008 | 0.060 |
| Kidney ³ | 0.128 | 0.206 | 0.207 | 0.988 |
| | ± | ± | ± | ± |
| | 0.008 | 0.024 | 0.033 | 0.090 |
| Lung ⁴ | 0.065 | 0.036 | 0.035 | 0.643 |
| | ± | ± | ± | ± |
| | 0.015 | 0.001 | 0.003 | 0.150 |
| Muscle | 0.000 | 0.000 | 0.000 | 1.230 |
| | | | | ± |
| | | | | 0.272 |
| Fat | 0.000 | 0.000 | 0.000 | 0.000 |
| Brain | 0.000 | 0.000 | 0.000 | 0.006 |
| | | | | ± |
| | | | | 0.001 |
| Plasma | 0.000 | 0.000 | 0.000 | 1.130 |
| | | | | ± |
| | | | | 0.837 |
| Urine ⁵ | 6.772 | 3.730 | 3.628 | 4.697 |
| | ± | ± | ± | ± |
| | 0.772 | 0.184 | 0.683 | 0.910 |

¹ n.s. for the comparison between L-Nys 2, 4, and 6 (Kruskal-Wallis ANOVA) and p = 0.01 for the comparison of L-Nys 2, 4, and 6 with D-AmB 1 (T-Test);
² n.s. and p = 0.09. 0.06, and 0.05;
³ n.s. and p = 0.01;
⁴ n.s. and 0.06, 0.05 and 0.05;
⁵ p = 0.05 and n.s.

The experimental design employed healthy New Zealand White rabbits weighing 3.0 to 3.2 kg. They were individually housed and maintained according to National Institutes of Health guidelines for laboratory animal care. Vascular access was established in each rabbit by placement of a subcutaneous silastic central venous catheter.

### EXAMPLE 1

### Urinary Pharmacokinetics

The urinary pharmacokinetics and drug disposition of liposomal nystatin (L-Nys; (Nyotran®, Aronex Pharmaceuticals, The Woodlands, Tex.)), amphotericin B deoxycholate (D-AmB; Fungizone®) were investigated in normal rabbits.

Four cohorts of 3 animals each received a single IV dose of L-Nys (2, 4, or 6 mg/kg) or D-AmB (1 mg/kg). Drug administration was by short intravenous infusion over 10 minutes following manual expression of the bladder under intravenous sedation. Plasma samples were obtained prior to dosing and for up to 24 hours after dosing using sparse sampling. Urine was collected in two-hour intervals for a total of 24 hours. Samples from all parenchymatous organs, muscle, and fat tissue were obtained at autopsy for determination of drug concentrations. Drug concentrations were determined by HPLC as nystatin and amphotericin B.

Pharmacokinetic parameters in plasma were derived using compartmental approaches with Bayesian estimation. The time points for sparse plasma sampling were determined using optimal sampling theory implemented by the ADAPT II computer program and full concentration-vs.-time profiles derived from prior pharmacokinetic studies (Lee et al. AAC 1994; 38:713; Groll et al AAC 2000; 44: 950, the teachings of which are incorporated herein by reference). In these studies, plasma profiles of nystatin after administration of the multilamellar liposomal formulation fitted best to a 2-compartment pharmacokinetic model, and the profiles of amphotericin B after administration of the deoxycholate formulation were best compatible with a 3-compartment pharmacokinetic model with intravenous bolus input and elimination from the central compartment. The selected time points for sparse sampling were Cmax, 30, 60 min. and 6 and 24 hours for nystatin, and Cmax, 15 min. and 2, 8 and 24 hours for amphotericin B, respectively.

Estimation of pharmacokinetic parameters in infected animals was based on the plasma concentration data at the selected optimal sampling time points and at 3 and 12 hrs and data from healthy rabbits as priors using maximum a priori likelihood and Bayesian estimation. The goodness of fit (R²) between estimated and observed data ranged from 0.998 and 1.000 for nystatin and 0.971 and 0.974 for amphotericin B, respectively.

AUC 0-24 h in urine and renal clearance were determined by standard techniques (Gibaldi & Perrier 1982, the teachings of which are incorporated herein by reference). The relative recovery of both compounds in body fluids and tissues 24 hours after administration of a single dose was calculated from total dose, drug concentrations, organ weights, blood volume, and the volume of 24 h urine collections, respectively. Recovery of compound from plasma was based on the assumption of a blood volume of 8 mL/kg and individually determined hematocrit values; recovery from skeletal muscle was based on a relative muscle mass of 50% of the total body weight, and recovery from fat tissue was based on a relative fat mass of 5% of the total body weight (Davies & Morris, Pharm Res 1993; 10: 1093, the teachings of which are incorporated herein by reference).

All values are presented as means of 3 animals each ±SEM. Differences between pharmaco-kinetic parameters of two cohorts were evaluated by Student's t-test and those between more than two cohorts were evaluated by the Kruskal-Wallis ANOVA. A two-tailed p-value of d0.05 was considered statistically significant. Pharmacokinetic parameters in urine and recoveries were calculated by standard techniques.

In comparison to a standard dose of D-AmB, urinary data (Table 2) revealed at least tenfold higher Cmax and at least fourfold higher AUC values after administration of L-Nys (p<0.001 by ANOVA; means/SEM). There was a trend (p =0.07; ANOVA) towards a dose-dependent decrease in the renal clearance of L-Nys, suggesting dose-dependent nephrotoxic effects or saturable tubular excretion. Independent of the dosage of L-Nys, at 24 hrs, less than 0.5% of the total dosage was recovered from liver, spleen, kidneys and lung (Tables 4 and 5). In contrast, after D-AmB, 25% of the dose was found in the liver. The recovery of L-Nys in urine decreased with increasing dosages (p=0.01; ANOVA), but was not significantly different from that of D-AmB, suggesting that the differences in the disposition of both compounds may be explained by fundamental differences in biliary excretion or hepatic metabolism.

The results established striking differences in the disposition of L-Nys and D-AmB. Liposomal nystatin and other polyenes are indicated in treatment of fungal infections of the urinary tract that require therapy with antifungal polyenes.

### EXAMPLE 2

### Treatment of Subject

An adult human female presents with a fungal infection (Candida albicans) in both kidneys. The subject is treated with doses of liposomal nystatin at 2 mg/kg for a minimum five day course of treatment. Urine is collected and cultured daily. Therapy is discontinued after 3 days of urine that contains no detectable fungal infection.

EXAMPLE 3

### Treatment of Subject

An adult immunocompromised male presents with a fungal infection (Torulopsis) in both kidneys. The subject is treated with doses of liposomal nystatin at 6 mg/kg for an initial 10 day course of treatment. Urine is collected and cultured daily. Dosage is reduced to 2 mg/kg at a point after 3 days of urine that contains no detectable fungal infection. Dosage at 2 mg/kg is chronically maintained.

Compositions of this invention are prepared by a number of methods. Reference is made to U.S. Pat. No. 5,178,875 to Lenk et al., “Liposomal-polyene preliposomal powder and method for its preparation” the teachings of which are incorporated herein by reference. The compositions of this invention possess valuable pharmacological properties in inhibiting fungal infection. This is done particularly by concentration in the renal and bladder areas by renal clearance. Of course, retrograde perfusion and introduction is also noted.

Several types of liposomes are useful. These generally comprise fatty substances such as phospholipids (pl), optionally cholesterol, and a polyene such as nystatin. Liposomes of the present invention comprising the nystatin and the phospholipid in a preferred Nys/pl weight ratio between about 0.01/10 and about 0.7/10, are noted with particular reference to a range being between about 0.02/1 0 and about 0.08/10. The Nys may be part of the phospholipid lamellae, part of the encapsulated intraliposomal fluid or both.

Preferred phospholipids of these liposomes include phosphatidylglycerol, phosphatidylcholine, sphingomyelin, phosphatidic acid or phosphatidylserine, the more preferred phospholipids being phosphatidylglycerol, phosphatidylcholine or a combination thereof. The most preferred phosphatidylglycerol is one consisting essentially of dimyristoylphosphatidylglycerol and the most preferred phosphatidylcholine is one consisting essentially of dimyristoylphosphatidylcholine.

When the liposomes of the present invention comprise dimyristoylphosphatidylglycerol and dimyristoylphosphatidylcholine note is made of a ratio between about 1-10 and 10-1, more particularly a ratio of about 3 to 7. The liposomes of the present invention may be multilamellar, unilamellar or have an undefined lamellar construction. A pharmaceutical composition comprising the liposomes of the present invention and a pharmaceutically acceptable carrier or diluent may be used for the therapy of disease conditions involving local or systemic fungal infections.

Thus, these compositions can be used fungal infections of the kidney, ureter and bladder. Administration is contemplated to include chronic, acute or intermittent regimens. The compositions are particularly useful in the treatment of renal fungal infections.

The compositions of this invention are generally administered to animals, including but not limited to mammals including humans.

The pharmacologically active compositions of this invention can be processed in accordance with conventional methods of Galenic pharmacy to produce medicinal agents for administration to patients, e.g., mammals including humans.

In some embodiments of the present invention, dosage forms include instructions for the use of such compositions.

For parenteral application, particularly suitable are injectable, sterile liposomal solutions. Ampules are convenient unit dosages.

Generally, the compositions of this invention are dispensed in dosages of from about 2 mg/kg/day to about 6 mg/kg/day fro from about 5 to about 10 or more days. Chronic dosing of 30 days or more is contemplated. Similarly, chronic-intermittent dosing is contemplated. Chronic-intermittent is about every other day or every third day for periods in excess of thirty days (fifteen doses).

It will be appreciated that the actual preferred amounts of active compositions in a specific case will vary according to the specific compositions being utilized, the particular compositions formulated, the mode of application, and the particular situs and organism being treated. Dosages for a given host can be determined using conventional considerations, e.g., by customary comparison of the differential activities of the subject compositions and of a known agent, e.g., by means of an appropriate, conventional pharmacological protocol.

1. A method of treating a urinary tract fungal infection in a human comprising systemically administering a therapeutically effective amount of a liposomal polyene.

2. The method of claim 1 wherein the polyene is a amphotericin B.

3. The method of claim 1 wherein the polyene is a nystatin.

4. The method of claim 3 wherein the therapeutically effective amount of liposomal polyene is from about 1 mg/kg to about 8 mg/kg/day.

5. The method of claim 1 wherein the therapeutically effective amount is a sub-MED amount.

6. The method of claim 5 wherein the polyene is nystatin and the sub-MED amount is from about 1 mg/kg to 1.5 mg/kg/day.

7. The method of claim 1 wherein administering is for at least about 5 days.

8. The method of claim 1 wherein the therapeutically effective amount is measured in peak plasma concentration of at least about 10 μg/mL.

9. The method of claim 8 wherein the peak plasma concentration of at least about 13 μg/mL.

10. The method of claim 1 wherein the therapeutically effective amount is measured in AUC at μg/mL/hr is at least about 29 μg/mL/hr.

11. The method of claim 10 wherein the AUC is at least about 52 μg/mL/hr.

12. The method of claim 1 wherein the therapeutically effective amount is as measured in glomerular filtrate.

13. A method of treating a urinary tract fungal infection in a human comprising systemically administering a therapeutically effective amount of a liposomal polyene wherein the fungal infection is selected from the group consisting of aspergillosis, candidiasis (e.g., C. parapsilosis, C. albicans, C. tropicalis, C. glabrata, C. lusitaniae), zygomycosis, cryptococcosis, histoplasmosis, blastomycosis, cladosporiosis, fusariosis, Bipolaris hawaiiensis, Dactylaria gallopava, torulopsosis, Acremonium kiliense, Cryptococcus neoformans, and Histoplasma capsulatum.
